(19) United States
(12) Patent Application Publication
(10) Pub. No.: US 2007/0264303 A1
(43) Pub. Date: Nov. 15, 2007

(54) Coating for medical devices comprising an inorganic or ceramic oxide and a therapeutic agent

(76) Inventors: Liliana Atanasoska, Edina, MN (US); Robert Warner, Woodbury, MN (US); Rick Gunderson, Maple Grove, MN (US); Jan Weber, Maple Grove, MN (US); Scott Schewe, Eden Prairie, MN (US)

Correspondence Address: JONES DAY, 222 EAST 41ST ST, NEW YORK, NY, 10017, US

(21) Appl. No.: 11/433,898

(22) Filed: May 12, 2006

(51) Int. Cl. A61F 2/00; A61K 33/24

(52) U.S. Cl. 424/423; 424/617; 514/291; 514/449

(57) ABSTRACT

The invention relates generally to an implantable medical device for delivering a therapeutic agent to the body tissue of a patient, and a method for making such a medical device. In particular, the invention pertains to an implantable medical device, such as an intravascular stent, having a coating comprising an inorganic or ceramic oxide, such as titanium oxide, and a therapeutic agent.

### FIELD OF THE INVENTION

The invention relates generally to an implantable medical device for delivering a therapeutic agent to the body tissue of a patient, and a method for making such a medical device. In particular, the invention pertains to an implantable medical device, such as an intravascular stent, having a coating comprising an inorganic or ceramic oxide, such as titanium oxide, and a therapeutic agent.

### BACKGROUND OF THE INVENTION

Medical devices have been used to deliver therapeutic agents locally to body tissue of a patient. For example, intravascular stents comprising a therapeutic agent have been used to locally deliver therapeutic agents to a blood vessel. Often such therapeutic agents have been used to prevent restenosis. Examples of stents comprising a therapeutic agent include stents that comprise a coating containing a therapeutic agent for delivery to a blood vessel. Studies have shown that stents having a coating with a therapeutic agent are effective in treating or preventing restenosis.

Even though medical devices having a coating with a therapeutic agent are effective in preventing restenosis, many coated medical devices, in addition to being coated with a therapeutic agent, are also coated with a polymer and use of such polymeric coatings may have disadvantages. For example, depending on the type of polymer used to coat the medical device, some polymers can cause inflammation of the body lumen, offsetting the effects of the therapeutic agent.

Also, some polymer coatings do not actually adhere to the surface of the medical device; instead the coatings encapsulate the surface, which makes the polymer coatings susceptible to deformation and damage during loading, deployment and implantation of the medical device. For instance, balloon expandable stents must be put in an unexpanded or “crimped” state before being delivered to a body lumen. The crimping process can tear the coating or cause the coating to be completely ripped off of the stent. Once in the crimped state the polymeric coating can cause adjacent stent surfaces, such as struts, to adhere to each other. Moreover, if the coating is applied to the inner surface of the stent, it may stick to the balloon as it contacts the inner surface during expansion. Such interference may prevent a successful deployment of the medical device.

Similarly to balloon-expandable stents, polymer coatings on self-expanding stents can also interfere with the deployment mechanism. Self-expanding stents are usually deployed using a pull back sheath system. When the system is activated to deploy the stent, the sheath is pulled back, exposing the stent and allowing the stent to expand itself. As the sheath is pulled back it slides over the outer surface of the stent. Polymer coatings located on the outer surface of the stent can adhere to the sheath as it is being pulled back and disrupt the deployment of the stent.

Any damage to the polymer coating may alter the drug release profile and which can lead to an undesirable and dangerous increase or decrease in the drug release rate.

Accordingly, there is a need for coatings for medical devices that have increased adhesion to the surface of a medical device. Moreover, there is a need for medical device coatings that are not easily deformed or damaged, particularly during loading, deployment or implantation of the medical device. There is also a need for coatings that have reduced tackiness so that undesired adhesion to the delivery system can be avoided.

### SUMMARY OF THE INVENTION

These and other objectives are accomplished by the present invention. The present invention, in one embodiment, provides a coating for a medical device, such as an intravascular stent. The coating comprises a therapeutic agent and an inorganic or ceramic oxide, such as titanium oxide. The inclusion of the inorganic or ceramic oxide enhances the adhesion of the coating to the medical device surface, especially when the surface is made of a material that is present in the inorganic or ceramic oxide. Also, if the medical device comprises a corrosive or non-biocompatible material, such as nickel, the inorganic or ceramic oxide coating can increase the biocompatibility of the medical device by preventing corrosion of the medical device as well as preventing undesirable materials from leaching out of the medical device.

One embodiment contemplated by the present invention is an implantable intravascular stent comprising: (a) a stent sidewall structure having a surface; and (b) a coating comprising a first metal oxide and a therapeutic agent disposed upon at least a portion of the surface, wherein the first metal oxide comprises a titanium oxide or an iridium oxide. In certain embodiments the first metal oxide can be a hydrophilic titanium oxide or a hydrophobic titanium oxide.

The surface of the stent sidewall structure of the stent can comprise nickel, titanium, nitinol, stainless steel or a combination thereof. Additionally, the coating can adhere to the surface of the medical device. Moreover, stent sidewalls of the present invention can comprise a plurality of struts and a plurality of openings. When the stent sidewall comprises a plurality of struts and a plurality of openings, the coating can conform to the surface to preserve the openings of the stent sidewall structure. Additionally, the stent can be a balloon-expandable stent or a self-expanding stent.

The first metal oxide can comprise about 1% to about 80% by weight of the coating or about 5% to about 30% by weight of the coating.

The therapeutic agent of the stent of the present invention, can comprise an anti-thrombogenic agent, anti-angiogenesis agent, anti-proliferative agent, antibiotic agent, an endothelial growth factor, immunosuppressant, radiochemical, or combination of thereof. Preferably, the therapeutic agent comprises an anti-restenosis agent or an endothelial growth factor. The therapeutic agent can also comprise paclitaxel, an analog thereof, a derivative thereof, or a conjugate thereof; sirolimus; tacrolimus; pimecrolimus; everolimus; or zotarolimus.

The therapeutic agent comprises about 1% to about 40% by weight of the coating or about 5% to about 30% by weight of the coating.

The coating can further comprise a polymer. The first metal oxide and the therapeutic agent can be dispersed in the polymer or, alternatively, the polymer and the therapeutic agent can be dispersed in the first metal oxide.

The polymer can comprise an a polyether, copolymers of Nylon 12 or Nylon 6 and polyethers (e.g. PEO or PTMO) such as, PEBAX, a polystyrene copolymer, a polyurethane, an ethylene vinyl acetate copolymer, a polyethylene glycol, a fluoropolymer, a polyaniline, a polythiophene, a polypyrrole, a maleated block copolymer, a polymethylmethacrylate, a polyethylenetheraphtalate or a combination thereof.

Also, the stent, of the present invention can further comprise a quantity comprising or consisting of an inorganic or ceramic oxide disposed between the surface and the coating. The inorganic or ceramic oxide can comprise a second metal oxide and, more specifically, the second metal oxide can comprise a titanium oxide or an iridium oxide.

Additionally, the coating can comprise a second inorganic or ceramic oxide. The second inorganic or ceramic oxide can comprise about 1% to about 30% by weight of the coating. The second inorganic or ceramic oxide can comprise a second metal oxide and, more specifically, the metal oxide can comprise a third titanium oxide or iridium oxide.

In another embodiment of the present invention, the present invention comprises an implantable intravascular stent comprising: (a) a balloon-expandable stent sidewall structure having a surface comprising a plurality of struts and a plurality of openings, wherein the stent sidewall structure comprises a metal; and (b) a coating comprising a titanium oxide and an anti-restenosis agent disposed upon and adhering to at least a portion of the surface, wherein the coating conforms to preserve the openings of the stent sidewall structure. The coating can further comprise a polymer. The stent sidewall structure can comprise stainless steel.

In another embodiment of the present invention, the invention comprises an implantable intravascular stent comprising: (a) a self-expanding stent having a sidewall structure having a surface comprising a plurality of struts and a plurality of openings, wherein the stent sidewall structure comprises nitinol; and (b) a coating comprising a titanium oxide and an anti-restenosis agent disposed upon and adhering to at least a portion of the surface. The coating can conform to the surface to preserve the opening of the stent sidewall structure. The coating can further comprise a polymer.

In another embodiment of the present invention, the invention comprises an embolic coil comprising: a coating comprising a titanium oxide and an anti-restenosis agent disposed upon and adhering to at least a portion of the surface. The coating can further comprise a polymer.

In yet another embodiment of the present invention, the present invention can be an implantable medical device comprising: (a) a surface; and (b) a coating comprising a first inorganic or ceramic oxide and a therapeutic agent disposed upon at least a portion of the surface. The coating can adhere to the surface. The surface can comprise of nickel, titanium, nitinol, stainless steel or a combination thereof.

Additionally, the first inorganic or ceramic oxide of the coating can comprise a metal oxide and the metal oxide can comprise titanium oxide, such as a hydrophilic titanium oxide or hydrophobic titanium oxide. The first inorganic or ceramic oxide comprises about 1% to about 80% by weight of the coating or about 5% to about 30% by weight of the coating.

The therapeutic agent can comprise an anti-thrombogenic agent, anti-angiogenesis agent, anti-proliferative agent, antibiotic agent, growth factor, immunosuppressant, radiochemical, or combination of thereof. Preferably, the therapeutic agent comprises an anti-restenosis agent. Suitable therapeutic agents include, but are not limited to, paclitaxel, an analog thereof, a derivative thereof, or a conjugate thereof; sirolimus; tacrolimus; pimecrolimus; everolimus; zotarolimus or. The therapeutic agent comprises about 1% to about 40% by weight of the coating or about 5% to about 30% by weight of the coating.

The coating can further comprise a polymer. The first inorganic or ceramic oxide and the therapeutic agent can be dispersed in the polymer or, alternatively, the polymer and the therapeutic agent can be dispersed in the first inorganic or ceramic oxide. Suitable polymers include, but are not limited to, a polyether, PEBAX, a polystyrene copolymer, a polyurethane, an ethylene vinyl acetate copolymer, a polyethylene glycol, a fluoropolymer, a polyaniline, a polythiophene, a polypyrrole, a maleated block copolymer, a polymethylmethacrylate, a polyethylenetheraphtalate or a combination thereof.

The implantable medical device can further comprise of a quantity comprising or consisting of an inorganic or ceramic oxide disposed between the surface and the coating. The inorganic or ceramic oxide can comprise a metal oxide and, more specifically, the metal oxide can be titanium oxide.

The coating can also comprise of a second inorganic or ceramic oxide. The second inorganic or ceramic oxide comprises about 1% to about 30% by weight of the coating. The second inorganic or ceramic oxide can comprise a second metal oxide and, more specifically, the metal oxide can be a second titanium oxide.

The present invention is also directed towards methods of making an implantable medical device comprising: (i) providing a medical device having a surface; and (ii) applying to at least a portion of the surface a coating composition to form a coating on the surface, wherein the coating composition comprises a inorganic or ceramic oxide and a therapeutic agent.

Preferably, the coating composition can be formed by a sol-gel process. The sol-gel process can be conducted at a temperature below the degradation temperature of the therapeutic agent. In one embodiment the sol-gel process is conducted at 200° C.

The coating composition of the methods of the present invention can comprise the steps of (i) preparing a precursor solution by dissolving an inorganic alkoxide in an organic solvent; (ii) adding an acid, base, water or a combination thereof to the precursor solution; (iii) allowing the precursor solution to undergo hydrolysis and condensation to form a gel.

The therapeutic agent can be added to the precursor solution before or after step (iii). Also, a polymer can be added to the precursor solution. The polymer can be added before or after step (iii).

Organic solvents can comprise an alcohol, ketone, toluene or a combination thereof. Suitable alcohols include, but are not limited to, isopropanol, hexanol, heptanol, octanol, methanol, ethanol, butanol or a combination thereof. Suitable ketones include, but are not limited to, methylethylketone. Suitable acids include, but are not limited to, acetic acid, citric acid, nitric acid or hydrochloric acid.

Additionally, the ratio of the inorganic or ceramic oxide to the alcohol can be between about 500:1 to 1:500, or between 400:1 to 1:400, or between 300:1 to 1:300, or between 200:1 to 1:200, or between 100:1 to 1:100, or between 50:1 to 1:50, or between 10:1 to 1:10. In certain embodiments the ratio of the inorganic or ceramic oxide to the alcohol is between about 1:6 to about 6:1. In other embodiments the ratio of the inorganic or ceramic oxide to the alcohol is between about 1:100 to about 1:300.

The coating composition of the methods of the present invention can further comprise exposing the coating to a heat treatment. The coating composition can be heated to a temperature of less than the degradation temperature of the therapeutic agent. In one embodiment the coating composition is heated to a temperature of less than about 200° C. The heat treatment can comprise a solvo-thermal treatment, a hydrothermal treatment, vacuum ultraviolet irradiation or a combination of the foregoing.

The therapeutic agent can comprise an anti-thrombogenic agent, anti-angiogenesis agent, anti-proliferative agent, antibiotic agent, anti-restenosis agent, endothelial growth factor, immunosuppressant, radiochemical, or combination thereof. Preferably, the therapeutic agent comprises an anti-restenosis agent or an endothelial growth factor. Suitable anti-proliferative agents include, but are not limited to, paclitaxel, analog thereof, derivative thereof, or conjugate thereof. Suitable therapeutic agents include, but are not limited to, sirolimus, tacrolimus, pimecrolimus or everolimus.

The inorganic alkoxide can comprise a metal alkoxide. Preferably, the metal alkoxide is a titanium alkoxide. Suitable titanium alkoxides include, but are not limited to, titanium butoxide, titanium tetraisopropoxide, titanium ethoxide or a combination of the foregoing.

The polymer can comprise a polyether, PEBAX, a polystyrene copolymer, a polyurethane, an ethylene vinyl acetate copolymer, a polyethylene glycol, a fluoropolymer, a polyaniline, a polythiophene, a polypyrrole, a maleated block copolymer, a polymethylmethacrylate, a polyethylenetheraphtalate or a combination thereof.

The methods of the present invention also include a method of making an implantable medical device for delivering a therapeutic agent to the body tissue of a patient, the method comprising: providing a medical device having a surface; and coating the surface with a coating composition, wherein the coating composition is formed by: (i) preparing a precursor solution by dissolving a titanium alkoxide in an organic solvent; (ii) adding an acid to the precursor solution; (iii) allowing the precursor solution to undergo hydrolysis and condensation to form a gel; (iv) adding a therapeutic agent to the precursor solution or the gel; and (v) heating the gel to a temperature less than 200° C.

### BRIEF DESCRIPTION OF THE DRAWINGS

The present invention will be explained with reference to the following drawings.

FIG. 1 shows a cross-sectional view of an embodiment of a coating disposed on at least a percent of a medical device.

FIG. 2 show a portions of a stainless steel surface that has been exposed to ion bombarment prior to coating.

FIG. 3 show a portions of a stainless steel surface that has been exposed to ion bombarment prior to coating.

FIG. 4 show a portions of a stainless steel surface that has been exposed to ion bombarment prior to coating.

FIG. 5 show a portions of a stainless steel surface that has been exposed to ion bombarment prior to coating.

FIG. 6 shows a cross-sectional view of another embodiment of a coating disposed on at least a portion of a medical device.

FIG. 7 shows a cross-sectional view of yet another embodiment of a coating disposed on at least a portion of a medical device.

FIG. 8 shows a layer of polymeric material disposed on the coating shown in FIG. 1.

FIG. 9 shows a medical device suitable for use in the present invention.

FIG. 10 shows a method for making a coated medical device of the present invention comprising a metal oxide.

FIG. 11 shows a method for making a coated medical device of the present invention comprising a titanium oxide.

FIG. 12 shows a titanium surface formed by using a sol-gel process.

FIG. 13 shows a titanium surface formed by using a sol-gel process.

FIG. 14 shows a titanium surface formed by using a sol-gel process.

FIG. 15 shows a titanium surface formed by using a sol-gel process.

FIG. 16 shows a titanium surface formed by using a sol-gel process.

### DETAILED DESCRIPTION

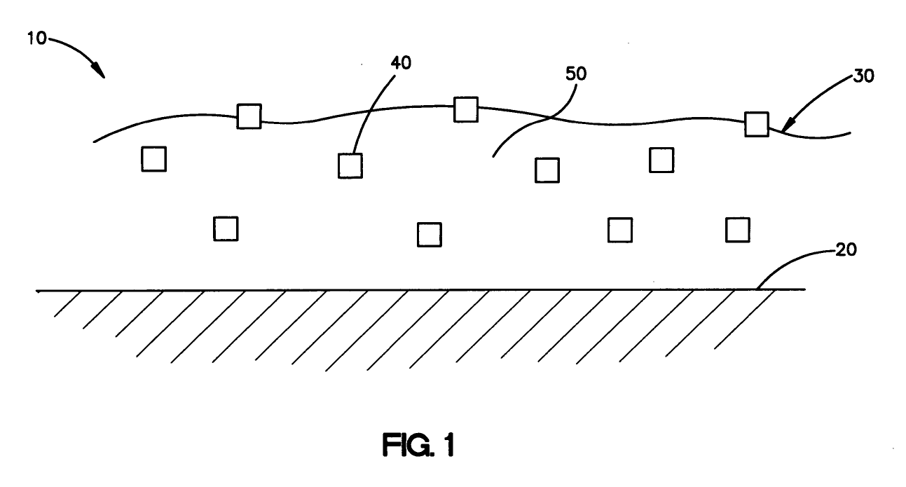

In one embodiment, the medical device of the present invention comprises a surface having a coating disposed thereon. The coating comprises an inorganic or ceramic oxide, such as a metal oxide like titanium oxide, and a therapeutic agent. FIG. 1 shows a cross-sectional view of an embodiment of a coating disposed on at least a portion of a surface of a medical device. In this embodiment, a medical device 10 has a surface 20. The medical device can be a stent and the surface can be the surface of a strut that makes up the stent. Disposed on at least a portion of the surface 20 is a coating 30. The coating 30 comprises an inorganic or ceramic oxide which in this embodiment is a metal oxide 50 and a therapeutic agent 40. In this embodiment, the therapeutic agent 40 is dispersed in the metal oxide 50. In alternate embodiments, the therapeutic agent can be dispersed in a matrix that includes the metal oxide as a component. Also, the coating can include more than one type of inorganic or ceramic oxide.

In certain embodiments, it is preferred that the inorganic material in the inorganic or ceramic oxide is the same as at least one material that is used to form the medical device or medical device surface. For instance, when the medical device surface is formed from a nickel and titanium alloy, such as nitinol, it may be preferable to have the metal oxide in the coating be a titanium oxide. Having a common metal in the coating and in the surface can increases adhesion of the coating to the surface.

However, the inorganic or ceramic oxide used in the coating need not have the same material used to form the medical device or medical device surface. For example, a coating comprising titanium oxide or silicon oxide can be used to coat a medical device made of stainless steel. If titanium oxide is used to coat stainless steel medical devices or other medical devices comprising stainless steel such as, MP35N, PERSS and Pt-SS, material for promoting adhesion of the coating can be used to create a mixed TiOx—SiOx coating. In certain embodiments silicone coupling agents can be added to the coating composition to promote adhesion of the coating to the surface of the medical device. Suitable silicon coupling agents include, but are not limited to, phenylethynyl imide silanes or isocyanatopropyl triethoxysilane.

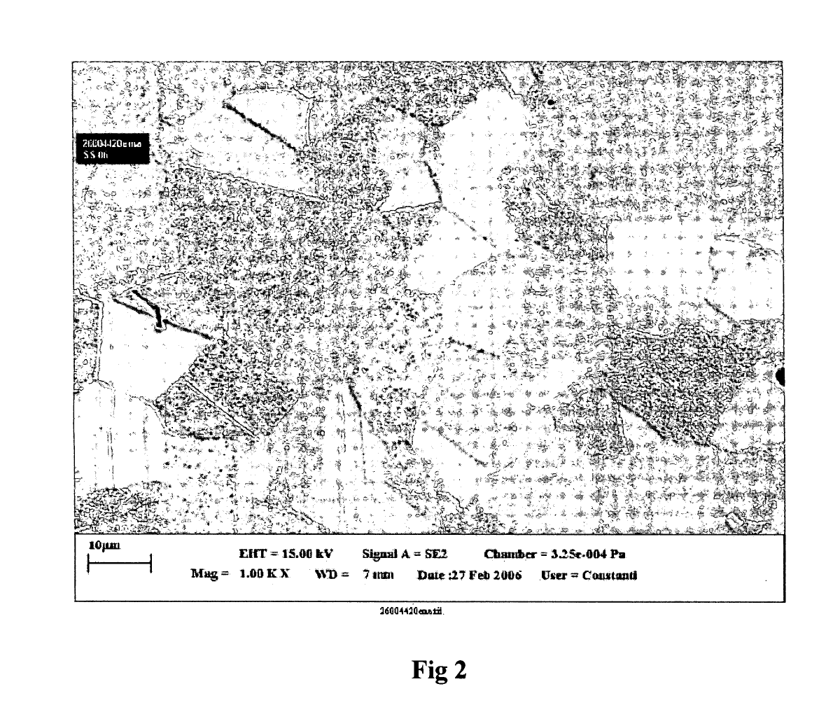

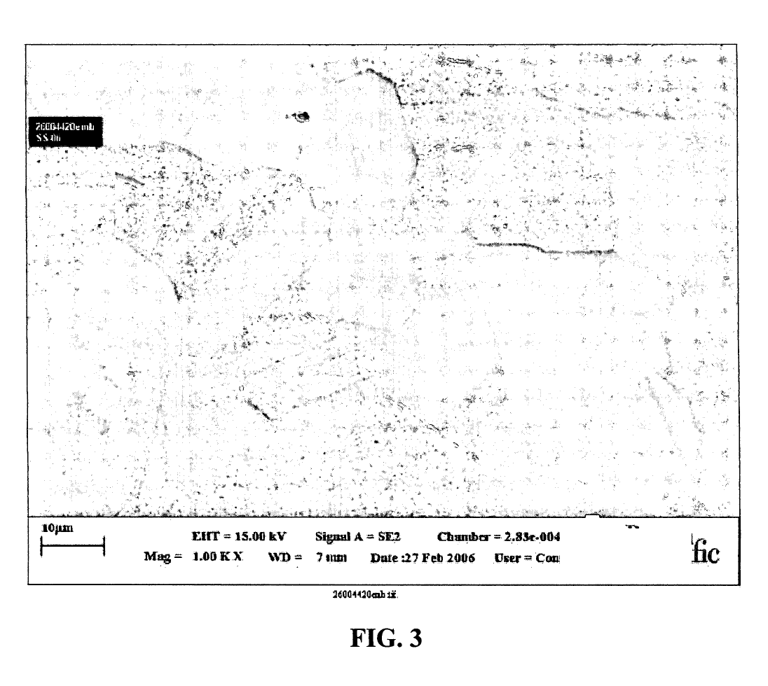

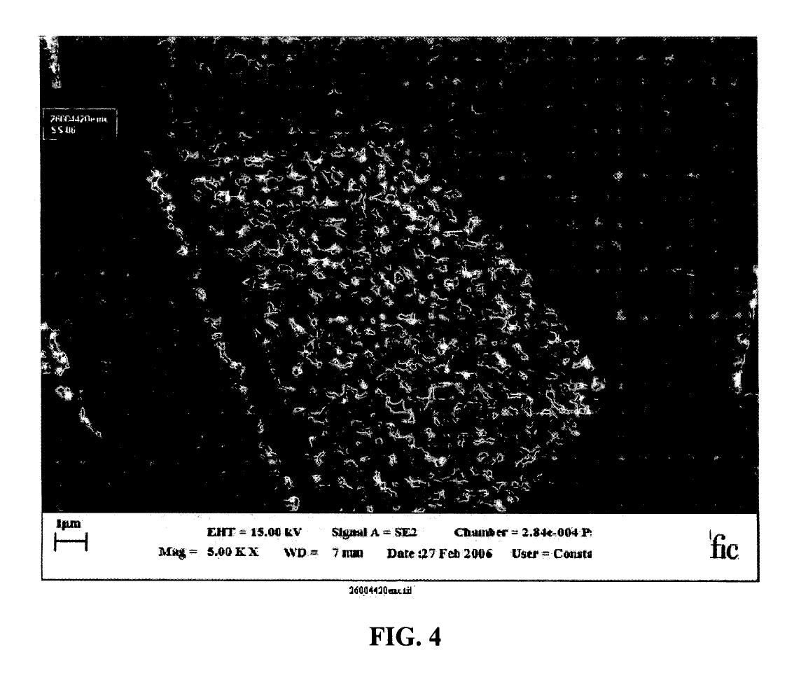

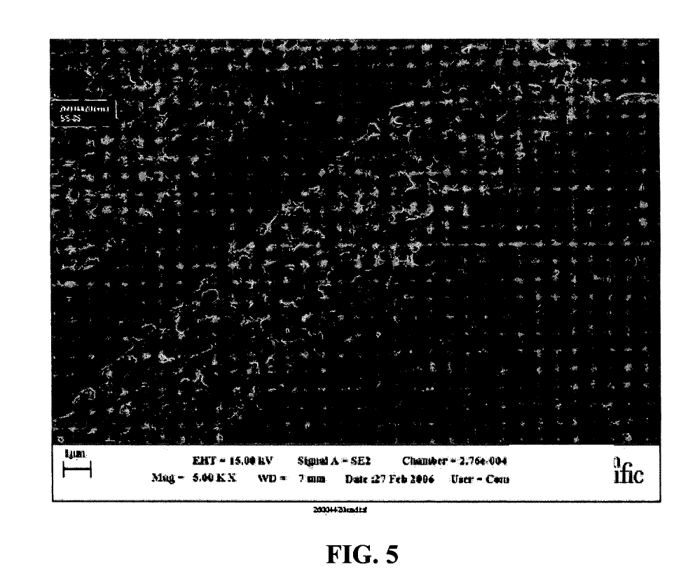

Additionally, if a stainless steel medical device is being coated with a coating comprising an inorganic or metal ceramic coating, the surface of the medical device can be treated with an argon ion implantation treatment, creating a nano-porous surface structure. FIG. 2 through FIG. 5 show a portions of a stainless steel, nano-porous surface that has been exposed to 4,000,000 pulses of 20×10¹⁷ argon ions/cm² at a frequency of 400 Hz in vacuum for two hours. Once the surface has been treated with an argon implantation treatment a titanium oxide layer can be applied to, or formed on the surface. The porous surface achieved by the argon ion implantation treatment is thought to improve the adherence of the titanium oxide coating. Alternatively, other inert elements such as helium can be used instead of argon to create a porous surface. The use of different inert element can be used to create different size pores.

Alternatively, following the Argon ion implantation treatment, the surface can potentially be treated with plasma vapor deposition of titanium or a titanium-carbon or titanium-nickel alloy and then coated with a coating comprising an inorganic or ceramic oxide and a therapeutic agent.

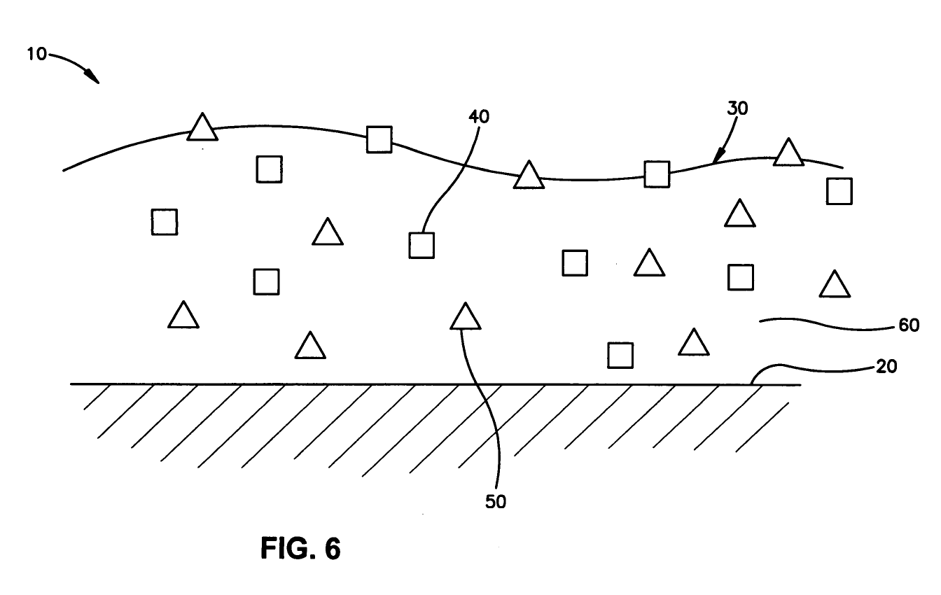

FIG. 6 shows a cross-sectional view of another embodiment of a coating disposed on at least a portion of a medical device. In this embodiment, a medical device 10 has a surface 20. Disposed on at least a portion of the surface 20 is a coating 30. The coating 30 comprises an inorganic or ceramic oxide 50, a therapeutic agent 40 and a polymer 60. In this embodiment, the therapeutic agent 40 and the inorganic or ceramic oxide 50 are dispersed in the polymer 60. In another embodiment, porous inorganic or ceramic nano or micro particles can be loaded with a therapeutic agent and then the porous metal oxide nano or micro particles can be dispersed in a polymer. Alternatively, the therapeutic agent and the polymer can be dispersed in the inorganic or ceramic oxide.

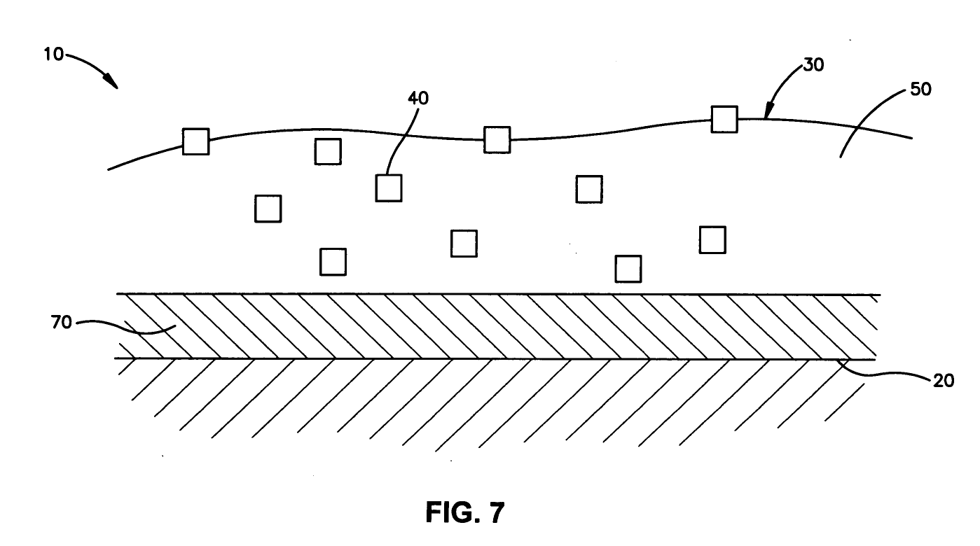

FIG. 7 shows a cross-sectional view of another embodiment. In this embodiment, a quantity of an inorganic or ceramic oxide 70 is disposed on at least a portion of a surface 20 of a medical device 10. The quantity of the inorganic or ceramic oxide 70 can be in the form of a layer. Disposed upon the quantity of inorganic or ceramic oxide 70 is a coating 30. The coating 30 comprises a second inorganic or ceramic oxide 50 and a therapeutic agent 40. The inorganic or ceramic oxide 70 disposed on the surface 20 can be the same as or different from the second inorganic or ceramic oxide 50 in the coating 30. In some embodiments, the quantity of inorganic or ceramic oxide 70 can consist of a metal oxide.

Suitable inorganic or ceramic oxides that can be included in the coating or disposed as a quantity or layer between the medial device surface and the coating can include ones where the inorganic material in the oxide is titanium, nickel, silicon, iron, platinum, tantalum, iridium, niobium, zirconium, tungsten, rhodium, cobalt, chromium, ruthenium.

Suitable inorganic or ceramic oxides include, without limitation, metal oxides such as, platinum oxide, tantalum oxide, titanium oxide, tantalum oxide, zinc oxide, iron oxide, magnesium oxide, aluminum oxide, iridium oxide, niobium oxide, zirconium oxide, tungsten oxide, rhodium oxide and ruthenium oxide; silicone oxides such as, silicon dioxide; inorganic-organic hybrids such as, titanium poly[(oligoethylene glycol)dihydroxytitanate]or combinations thereof.

In some embodiments, it is preferred that the metal oxide be a titanium oxide. Examples of suitable titanium oxides include without limitation, titanium dioxide, titanium butoxide, titanium tetraisopropoxide and titanium ethoxide.

The phrase “titanium oxide” as used herein comprises titanium of various valence states, such as, lower valence state titanium oxide with Magneli structure for lubriciousness; other crystalographic forms of titanium oxide, such as, anatase and rutile; inorganic-organic hybrids, including polyethylene glycol one, such as, titanium poly[(oligoethylene glycol)dihydroxytitanate].

In some embodiments, the inorganic or ceramic or metal oxide comprises at least 5%, at least 10%, at least 20%, at least 30%, at least 40%, at least 50%, at least 60%, at least 70%, at least 80%, at least 90%, at least 95%, at least 97%, at least 99% or more by weight of the coating. Preferably, the inorganic or ceramic or metal oxide is about 1% to about 80% by weight of the coating. More preferably, the therapeutic agent is about 5% to about 30% by weight of the coating.

The coating may be of any thickness. In some embodiments, the coating preferably has a thickness of about 1 to about 10 microns or, more preferably, about 2 to about 5 microns. In some instances, a relatively thicker film may be preferred to incorporate greater amounts of the therapeutic agent. In addition, a relatively thicker film may allow the therapeutic agent to be released more slowly over time. The coating can also have a uniform distribution of pores, therapeutic agents or both. Additionally, if the coating further comprises a polymer, the coating can have a uniform distribution of the polymer.

In another embodiment of the present invention a polymeric material can be disposed over at least a portion of the coating. The polymeric material, which may be in the form of a layer, is disposed on the coating and can be used to control or regulate the release of the therapeutic agent from the coating. For instance such a layer of polymeric material may be disposed over any of the embodiments shown in FIGS. 1, 6 and 7. The layer of polymeric material can be of any thickness. In certain embodiments, the layer of polymeric material preferably has a thickness of about 1 to about 10 microns. Also, the polymeric material layer may also comprise a therapeutic agent that may be the same as or different from the therapeutic agent in the coating.

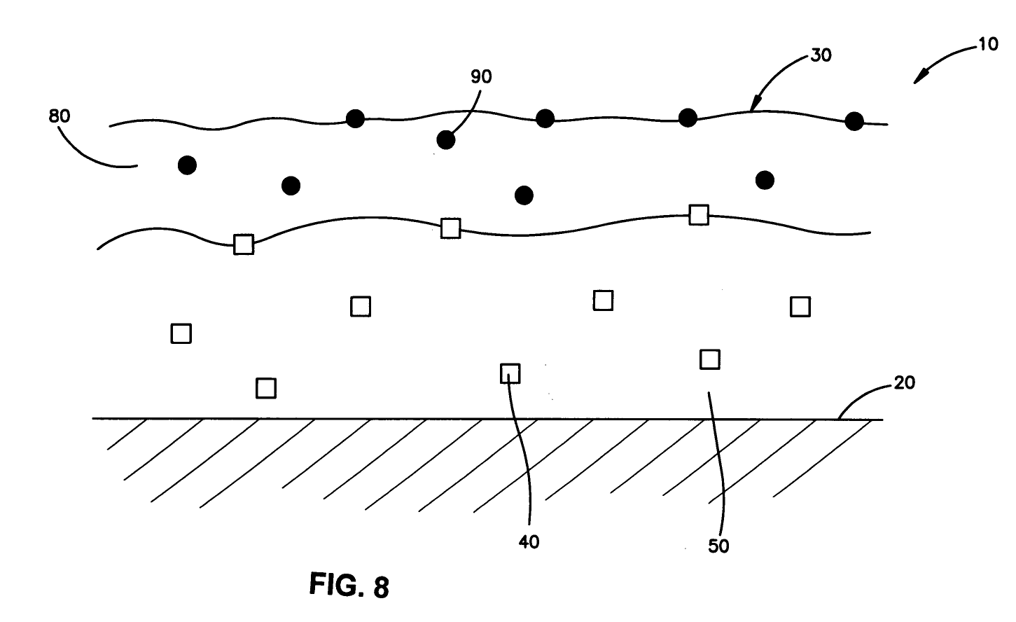

FIG. 8 shows a layer of a polymeric material 80 disposed upon the coating shown in FIG. 1. In FIG. 8, the polymeric material layer 80 includes a therapeutic agent 90 that is different from the therapeutic agent 40 of the coating 30.

### A. Medical Devices

Suitable medical devices for the present invention include, but are not limited to, stents, surgical staples, cochlear implants, embolic coils, catheters, such as central venous catheters and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, extra-corporeal devices such as blood oxygenators, blood filters, hemodialysis units, hemoperfusion units or plasmapheresis units.

Medical devices which are particularly suitable for the present invention include any stent for medical purposes, which are known to the skilled artisan. Suitable stents include, for example, vascular stents such as self-expanding stents, balloon expandable stents and sheet deployable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. In preferred embodiments, the stent suitable for the present invention is an Express stent. More preferably, the Express stent is an Express™ stent or an Express2™ stent (Boston Scientific, Inc. Natick, Mass.).

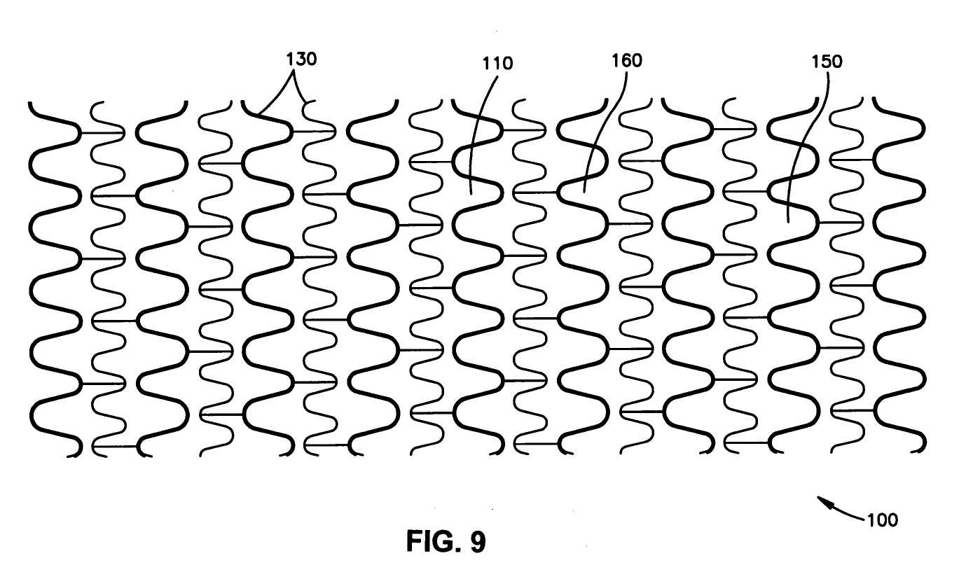

FIG. 9 shows an example of a medical device that is suitable for use in the present invention. This figure shows an implantable intravascular stent 100 comprising a sidewall 110 which comprises a plurality of struts 130 and at least one opening 150 in the sidewall 110. Generally, the opening 150 is disposed between adjacent struts 130. Also, the sidewall 110 may have a first sidewall surface 160 and an opposing second sidewall surface, which is not shown in FIG. 8. The first sidewall surface 160 can be an outer sidewall surface, which faces the body lumen wall when the stent is implanted, or an inner sidewall surface, which faces away from the body lumen wall. Likewise, the second sidewall surface can be an outer sidewall surface or an inner sidewall surface. In a stent having an open lattice sidewall stent structure, in certain embodiments, it is preferable that the coating applied to the stent conforms to the surface of the stent so that the openings in the stent structure is preserved, e.g. the openings are not entirely or partially occluded with coating material.

The framework of the suitable stents may be formed through various methods as known in the art. The framework may be welded, molded, laser cut, electro-formed, or consist of filaments or fibers which are wound or braided together in order to form a continuous structure.

Medical devices that are suitable for the present invention may be fabricated from metallic, ceramic, polymeric or composite materials or a combination thereof. Preferably, the materials are biocompatible. Metallic material is more preferable. Suitable metallic materials include metals and alloys based on titanium (such as nitinol, nickel titanium alloys, thermo-memory alloy materials); stainless steel; tantalum, nickel-chrome; or certain cobalt alloys including cobalt-chromium-nickel alloys such as Elgiloy® and Phynox®; PERSS (Platinum EnRiched Stainless Steel) and Niobium. Metallic materials also include clad composite filaments, such as those disclosed in WO 94/16646. Preferred, metallic materials include, platinum enriched stainless steel and zirconium and niobium alloys.

Suitable ceramic materials include, but are not limited to, oxides, carbides, or nitrides of the transition elements such as titanium, hafnium, iridium, chromium, aluminum, and zirconium. Silicon based materials, such as silica, may also be used.

Suitable polymeric materials for forming the medical devices may be biostable. Also, the polymeric material may be biodegradable. Suitable polymeric materials include, but are not limited to, styrene isobutylene styrene, polyetheroxides, polyvinyl alcohol, polyglycolic acid, polylactic acid, polyamides, poly-2-hydroxy-butyrate, polycaprolactone, poly(lactic-co-clycolic)acid, and Teflon.

Polymeric materials may be used for forming the medical device in the present invention include without limitation isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for medical devices include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

Medical devices may also be made with non-polymeric materials. Examples of useful non-polymeric materials include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Non-polymeric materials may also include biomaterials such as stem sells, which can be seeded into the medical device prior to implantation. Preferred non-polymeric materials include cholesterol, glyceryl monostearate, glycerol tristearate, stearic acid, stearic anhydride, glyceryl monooleate, glyceryl monolinoleate, and acetylated monoglycerides.

### B. Therapeutic Agents

The term “therapeutic agent” as used in the present invention encompasses drugs, genetic materials, and biological materials and can be used interchangeably with “biologically active material”. In one embodiment, the therapeutic agent is an anti-restenotic agent. In other embodiments, the therapeutic agent inhibits smooth muscle cell proliferation, contraction, migration or hyperactivity. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), pimecrolimus, amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifinctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, paclitaxel (as well as its derivatives, conjugates (including polymer deriviatives), analogs or paclitaxel bound to proteins, e.g. Abraxane™) 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs, conjugates (including polymer conjugates) or derivatives. Examples of polymer-drug conjugates are described in J. M. J. Frechet, Functional Polymers: Form Plastic electronics to Polymer-Assisted Therapeutics, 30 Prog. Polym. Sci. 844 (2005), herein incorporated by reference in its entirety. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other Non-Genetic Therapeutic Agents Include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, zotarolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, paclitaxel conjugates and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, paclitaxel 2-N-methypyridinium mesylate, and 2′-O-ester with N-(dimethylaminoethyl)glutamide hydrochloride salt. Paclitaxel conjugates suitable for use in the present invention include, paclitaxel conjugated with docosahexanoic acid (DHA), paclitaxel conjugated with a polyglutimate (PG) polymer and paclitaxel poliglumex.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol, glycosides, tacrolimus, pimecrolimus and zotarolimus.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

In some embodiments, the therapeutic agent comprises at least 5%, at least 10%, at least 20%, at least 30%, at least 40%, at least 50%, at least 60%, at least 70%, at least 80%, at least 90%, at least 95%, at least 97%, at least 99% or more by weight of the coating. Preferably, the therapeutic agent is about 1% to about 40% by weight of the coating that contains the therapeutic agent. More preferably, the therapeutic agent is about 5% to about 30% by weight of the coating that contains the therapeutic age.

### C. Polymers

Polymers useful for forming the coatings should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers, ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, and polylactic acid-polyethylene oxide copolymers.

When the polymer is being applied to a part of the medical device, such as a stent, which undergoes mechanical challenges, e.g. expansion and contraction, the polymers are preferably selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. The polymer is selected to allow the coating to better adhere to the surface of the strut when the stent is subjected to forces or stress. Furthermore, although the coating can be formed by using a single type of polymer, various combinations of polymers can be employed.

Examples of suitable hydrophobic polymers or monomers include, but not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymeric materials characterized by repeating siloxane groups, represented by Ra SiO 4-a/2, where R is a monovalent substituted or unsubstituted hydrocarbon radical and the value of a is 1 or 2; and naturally occurring hydrophobic polymers such as rubber.

Examples of suitable hydrophilic polymers or monomers include, but not limited to; (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl(meth)acrylate and 2-hydroxypropyl(meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO3). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Other suitable polymers include without limitation: polyurethanes, silicones (e.g., polysiloxanes and substituted polysiloxanes), and polyesters, styrene-isobutylene-copolymers. Other polymers which can be used include ones that can be dissolved and cured or polymerized on the medical device or polymers having relatively low melting points that can be blended with therapeutic agents. Additional suitable polymers include, but are not limited to, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, polyether block amides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluoropolymers, fluorosilicones, polyethylene glycol, polysaccharides, phospholipids, and combinations of the foregoing.

In certain embodiments block-copolymers are preferred for their ability to help create mesostructured and/or mesoporous coatings. For example, block-copolymers with both hydrophilic and hydrophobic components can create mesostructured of mesoporous coatings by organizing the coating components according to hydrophobicity and hydrophilicity. In certain embodiments preferred polymers include, but are not limited to, a polyether, Nylon and polyether copolymers such as PEBAX, a polystyrene copolymer, a polyurethane, an ethylene vinyl acetate copolymer, a polyethylene glycol, a fluoropolymer, a polyaniline, a polythiophene, a polypyrrole, a maleated block copolymer, a polymethylmethacrylate, a polyethylenetheraphtalate or a combination thereof.

### D. Methods of Making Coatings

To make the medical device of the present invention, a coating composition comprising the inorganic or ceramic oxide is used to form the coating. The coating composition can be formed by a sol-gel process or by making an inorganic or ceramic oxide suspension.

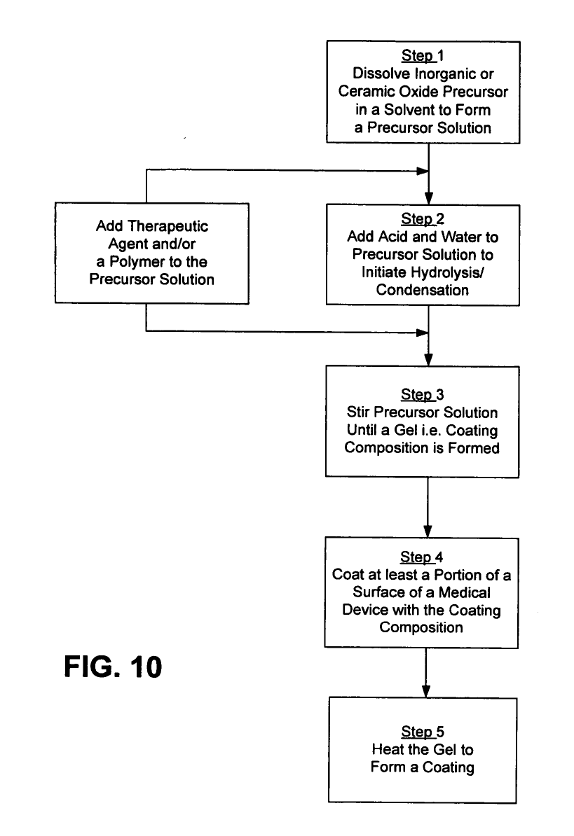

Sol-gel processes involve the formation of a colloidal suspension, i.e., the sol, and gelation of the sol to form a network in a continuous liquid phase, i.e., the gel. A general description of a sol-gel process suitable for the present invention is shown in FIG. 10.

In general, the sol-gel process begins with the making of a precursor solution or sol, as shown in Step 1 of FIG. 10. Precursor solutions can be made by dissolving a precursor in an alcohol or other organic solvent system. The precursor can be added drop-wise to the alcohol or other organic solvent while being continuously stirred. Generally, the precursor solution is stirred at room temperature; however, the solution can be stirred at high temperatures so long as the components of the precursor solution do not degrade. Surfactants and complexing agents can also be added to the precursor solution in order to help the precursor dissolve. In certain embodiments the surfactants are charged surfactants i.e. pluronic, anionic or cationic surfactants. Surfactants can be used, in addition to stabilizing solutions, to tailor the release of the therapeutic agent. The types of surfactant used will depend on the therapeutic agent used in the coating as well as the desired release profile.

Once the precursor solution is formed, water, an acid, a base or a combination thereof can be added to initiate hydrolysis and condensation, as shown in Step 2 of FIG. 10. The water, acid or base can be added at room temperature. A solution or suspension of the therapeutic agent can be added to the precursor solution before or after initiation of hydrolysis and condensation. Also, if a polymer is being used in the coating, the solution or suspension of the polymer can be added to the precursor solution before or after initiation of hydrolysis and condensation.

As shown in Step 3 of FIG. 10, the precursor solution is then stirred continuously until a gel is formed. The stirring can generally occur up to 24 hours at room temperature. Once the gel is formed the coating composition is applied to at least a portion of a surface of a medical device, as shown in Step 4 of FIG. 10.

Optionally, the precursor solution can be heated prior to being coated on the surface of a medical device in order to facilitate hydrolysis and condensation. For example the precursor solution can be placed under refluxing conditions or placed in an oven. The temperature and the length of time that the precursor solution is heated, depends on the composition of the precursor solution.

After the coating composition is applied to at least a portion of a surface of a medical device, the coating composition is heated as required for aging and removal of organic solvents. Aging is an extension of the formation of the gel in which the gel network is reinforced through further polymerization. Aging allows for densification of the coating and/or to achieve desired drug release properties.

Suitable heat treatments include, low temperature treatments, for example, solvo-thermal treatments, hydrothermal treatments, microwave treatments or vacuum ultraviolet irradiation. Again, the temperature, at which the coating is heated, depends on the composition of the coating composition. For example, if the coating composition comprises a therapeutic agent then the coating composition should not be heated to or beyond a temperature that would cause the therapeutic agent to degrade. Additionally, heat treatments such as ultraviolet radiation can be used to tailor the hydrophilic and hydrophobic properties of the inorganic or ceramic material, such as, titanium oxide. Therefore, the inorganic or ceramic coating can be tailored to accommodate either hydrophilic or hydrophobic therapeutic agents. Additional examples of suitable sol-gel processes are described in Zhijian Wu et al., “Design of Doped Hybrid Xerogels for a Controlled Release of Brillian Blue FCF”, 342 Journal of Non-Crystalline Solids 46 (2004), incorporated herein by reference in its entirety.

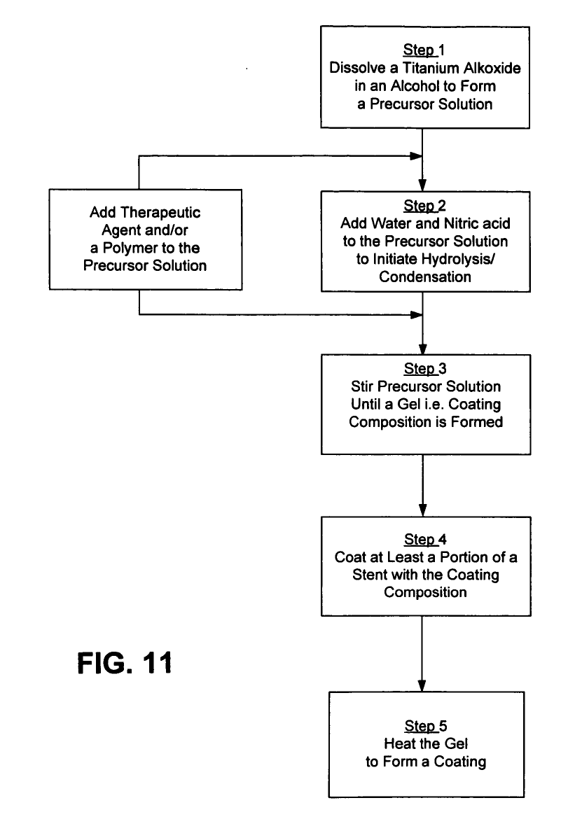

FIG. 11 shows a flow chart that further describes a sol-gel process for making a coating composition with a titanium alkoxide (TiOR₄), in accordance with the present invention. This process begins with preparing a precursor solution by dissolving a titanium alkoxide (TiOR₄) in dehydrated alcohol, as shown in Step 1 of FIG. 11. The titanium alkoxide (TiOR₄) can be added drop-wise to the dehydrated alcohol while being continuously stirred at room temperature. The volume ratio of the inorganic or ceramic oxide to the alcohol can be between about 500:1 to about 1:500, or between about 400:1 to about 1:400, or between about 300:1 to about 1:300, or between about 200:1 to about 1:200, or between about 100:1 to about 1:100, or between about 50:1 to about 1:50, or between about 10:1 to about 1:10. In certain embodiments the ratio of the inorganic or ceramic oxide to the alcohol is between about 1:6 to about 6:1. In other embodiments the ratio of the inorganic or ceramic oxide to the alcohol is between about 1:100 to about 1:300.

A required stoichometric amount of distilled water and nitric acid can be added at room temperature to initiate hydrolysis and condensation, as shown in Step 2 of FIG. 11. A solution of therapeutic agent, such as paclitaxel, can be added before or after initiation of hydrolysis and condensation reaction. Also, a polymer can be added to the precursor solution before or after initiation of hydrolysis and condensation.

The precursor solution can be stirred, at room temperature, for up to 24 hours or until a gel is formed, as shown in Step 3 of FIG. 11. The resulting gel or coating composition is then applied to at least a portion of a medical device, such as a stent.

The coating composition is then heated, as shown in Step 4 of FIG. 11. The coating composition should not be heated above the temperature at which the therapeutic agent begins to degrade. For example, paclitaxel degrades at a temperature of about 200° C. Therefore a coating composition containing paclitaxel should be heated to a temperature of less than 200° C. In an alternative embodiment, a precursor solution can include a titanium alkoxide in combination with an isocyanate functionalized alkoxy silane dissolved or suspended in an alcohol or other suitable organic solvent.

Suitable heat treatments include, low temperature treatments, for example, solvo-thermal treatments, hydrothermal treatments, microwave treatments or vacuum ultraviolet irradiation. The heat treatment can be applied for up to 20 hours or as required for aging, removal of organic residues and/or until the desired drug release properties are obtained. Preferably the heat treatment does not heat the coating composition to a temperature that would adversely affect the therapeutic agent, i.e., cause it to degrade.

The coating composition can be applied by any method known in the art. Examples of suitable methods include, but are not limited to, spray-coating such as by conventional nozzle or ultrasonic nozzle, dipping, rolling, electrostatic deposition, spin-coating or batch processes, such as air suspension, pan coating, ultrasonic mist spraying or ink-jet printing.

For the above sol-gel process, suitable precursors include, but are not limited to, inorganic alkoxides, metal acetates, metal salts of short and long chain fatty acids (e.g. hexanoate, octanoate, neodecanoate), metal salts of acetyl acetonate and peroxo titanium precursors.

Inorganic alkoxides include, but are not limited to, metal alkoxides such as titanium alkoxides; semi-metal alkoxides such as alkoxy silanes; or a combination of the forgoing.

Suitable titanium alkoxides include, but are not limited to, titanium butoxide, titanium tetraisopropoxide and titanium ethoxide.

Suitable alkoxy silanes include but are not limited to, isocyanate functionalized alkoxy silanes, tetraethoxysilane, methyltriethoxysilane, vinyltriethoxysilane, propyltriethoxysilane, phenyltriethoxysilane.

In one embodiment the precursor comprises isocyanate finctionalized alkoxy silanes in combination with titanium alkoxides.

For the above sol-gel process, suitable organic solvents include, but are not limited to, alcohols, such as isopropanol, hexanol, heptanol, octanol, methanol, ethanol, butanol, ketones, such as methylethylketons, toluene, or a combination thereof.

The release profile of the therapeutic agent from the coating can be adjusted by altering the sol-gel synthesis parameters, i.e., adjusting the pH, adjusting the water to alkoxide ratio, adjusting the heat time and temperature, changing the type of precursor, such as the type of titanium alkoxide. Additionally, dopants can be added during the process. Dopants can be used to introduce pores in to the coating, affecting the release profile of the therapeutic agent. Dopants may include sodium dodecyl sulfate, hydroxypropyl cellulose or cetyltrimethylammonium bromide.

The methods of the present invention also encompass methods of forming a coating using sol-gel processes that do not restrict heating to low temperatures. In certain embodiments, a precursor solution can be made by dissolving a precursor in an alcohol or other organic solvent system. The precursor can be added drop-wise to the alcohol or other organic solvent while being continuously stirred. Once the precursor solution is formed, water, an acid, a base or a combination thereof can be added to initiate hydrolysis and condensation.

The precursor solution is then stirred continuously until a gel is formed. Once the gel is formed the gel is applied to at least a portion of a surface of a medical device and is heated as required for aging and removal of organic solvents, creating a coating comprising an inorganic or ceramic material. Since the gel does not comprise a therapeutic agent or a polymer the gel coating can be heated to a high temperature. Once the surface has been coated with the inorganic or ceramic material, a therapeutic agent or a therapeutic agent and a polymer can then be applied to the medical device or, alternatively, an additional layer containing an inorganic or ceramic material alone or in addition to the therapeutic agent or the therapeutic agent and polymer can then be applied.

The gel can be applied by any methods commonly known in the art such as spray coating, dipping, rolling and ink-jet printing. Ink-jet printing is preferred when it is desired to apply the gel in a pattern such as stripes or dots.

In other embodiments, an aqueous suspension of inorganic or ceramic oxide particles and a therapeutic agent is formed and applied to the surface of a medical device. The suspension can be formed by first forming inorganic or ceramic oxide micro or nano-particles using a sol-gel process wherein precursor solution is made by dissolving a precursor in an alcohol or other organic solvent system, as discussed above. The precursor solution is then stirred and heated, preferably with microwaves, until inorganic or ceramic oxide micro or nano-particles are formed. A therapeutic agent can then be added to the inorganic or ceramic oxide micro or nano-particles. The inorganic or ceramic oxide micro or nano-particles and the therapeutic agent are then dispersed through a polymer/solvent solution creating a suspension. The suspension is then applied to the surface of a stent. The suspension can be any methods known in the art such as dip-coating.

In this embodiment preferred inorganic or ceramic oxides include, but are not limited to, titanium oxide. Additionally, preferred therapeutic agents include, but are not limited to, polar therapeutic agents such as, conjugated paclitaxel, heparin or an encapsulated hydrophobic drug in a polyionic shell.

In addition to sol-gel processes, the present invention also encompasses other methods if making a coating for a medical device, such as an intravascular stent wherein the coating comprises a therapeutic agent and an inorganic or ceramic oxide, such as titanium oxide. Such methods comprise making a coating composition comprising dispersing inorganic or ceramic oxide nano or micro size particles, not made by a sol-gel process, into a polymeric material and applying the coating composition to at least a portion of a surface of a medical device. Additionally, a therapeutic agent can also be dispersed in the polymer and inorganic or ceramic oxide coating composition. Suitable methods for dispersing nano or micro size particle in polymeric material in taught in U.S. Pat. No. 6,803,070 to Weber, which is herein incorporated by reference in its entirety.

In an alternative embodiment the method comprises making a coating composition comprising combining inorganic or ceramic oxide nano or micro size particles and a monomer; applying the coating composition to at least a portion of a surface of a medical device and polymerizing the monomer.

The medical devices and stents of the present invention may be used for any appropriate medical procedure. Delivery of the medical device can be accomplished using methods well known to those skilled in the art.

The following examples are for purposes of illustration and not for purposes of limitation.

### EXAMPLE 1

Sample coatings A through E comprising PEBAX (a copolymer of Nylon 12 or Nylon 6 and polyethers) and titanium were formed on stainless steel coupons. In sample coatings A through E titanium tetraisopropoxide, triethoxysilylpropylisocyanate and combinations thereof where used as precursors. PEBAX was the polymer used. The weight percentages of the precursors PEBAX used in coatings A through E are shown in Table 1.

TABLE 1

| | Titanium | | |
|---|---|---|---|
| Sample | Tetraisopropoxide | 3-triethoxysilylpropylisocyanate | PEBAX |
| A | 1% | 1% | 1% |
| B | 1% | 0.5% | 0.5% |
| C | 0.5% | 0.5% | 0.5% |
| D | 1% | 0 | 0.5% |
| E | 0.5% | 0 | 0.5% |

Titanium tetraisopropoxide, triethoxysilylpropylisocyanate or a combination is dissolved in a suitable organic solvent system and is added to a solution of butanol and PEBAX under stirring conditions at 60° C. An HCl aqueous solution is added in order to keep the water to titanium tetraisopropoxide molar ratio to 2:1. Once the hydrolysis is complete, the coating composition is continuously stirred for about 6.5 hours at 60° C. or for as long as necessary for aging.

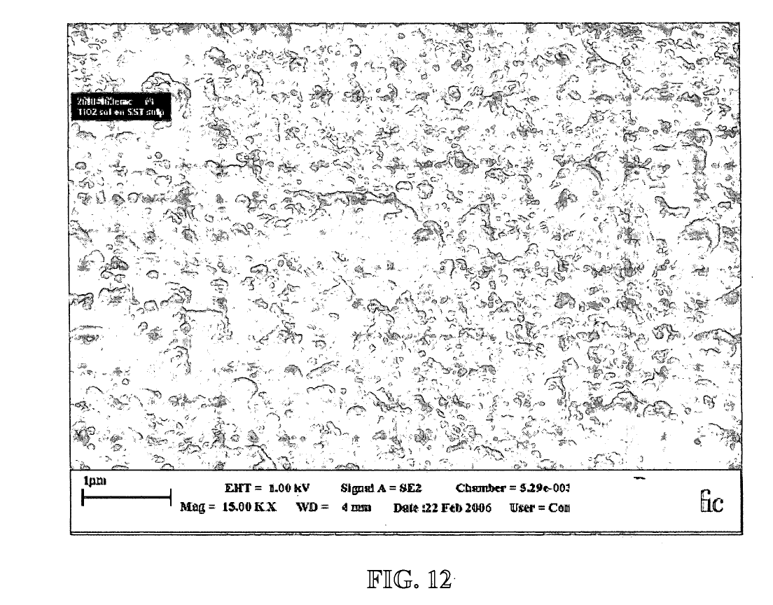

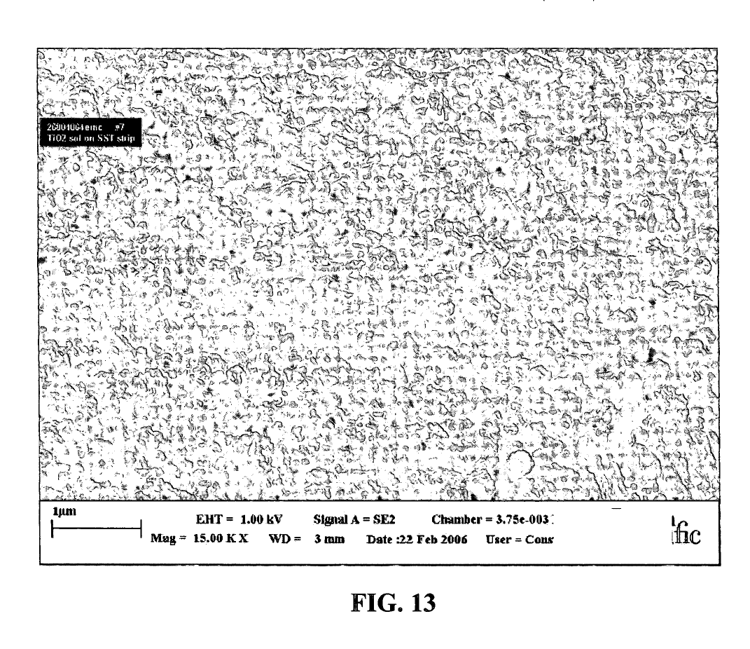

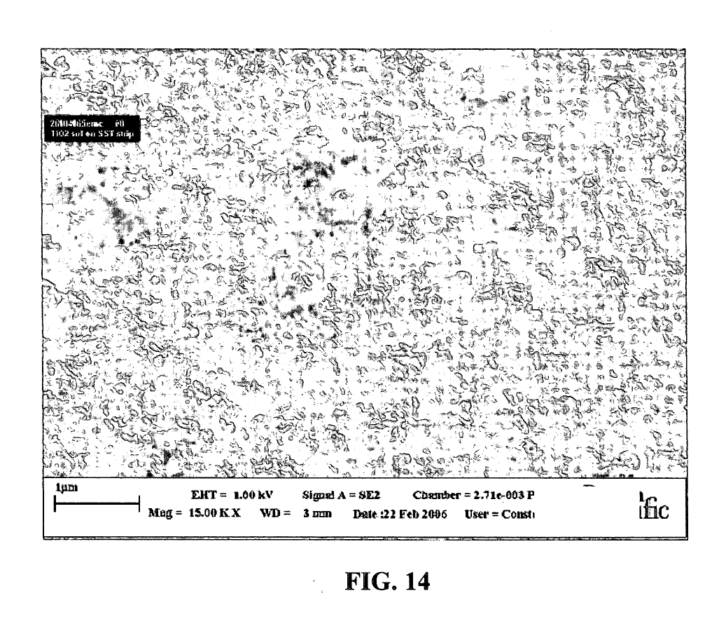

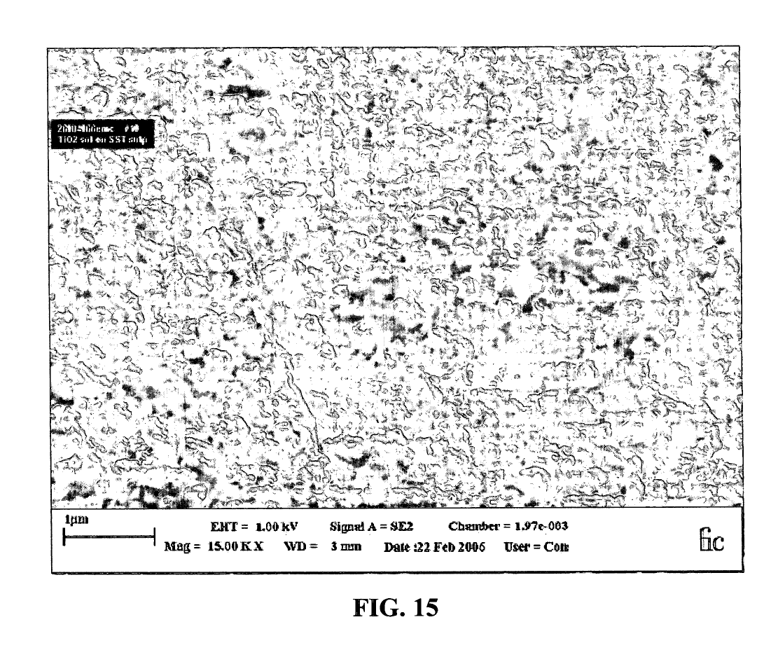

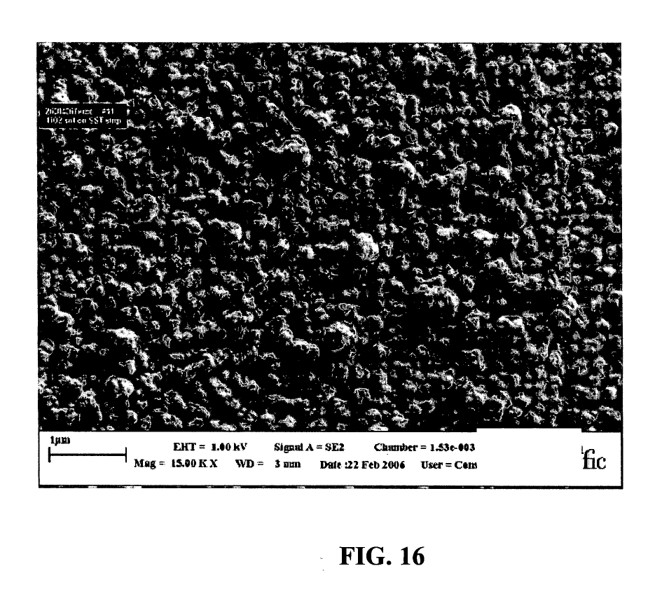

The coating composition is then applied to the surface of stainless steel coupons. The coated coupons were heated at 540° C. for about 2 hours to burn off the polymer and change the phase of the titania from brookite to anatase. FIGS. 12-16 show the resulting coating at 15,000× magnification.

### EXAMPLE 2

Titanium tetraisopropoxide is dissolved in a suitable organic solvent system and is added to a solution of butanol and PEBAX (a copolymer of Nylon 12 or Nylon 6 and polyethers) under stirring conditions at 60° C. An HCl aqueous solution is added in order to keep the water to titanium tetraisopropoxide molar ratio to 2:1. Once the hydrolysis is complete, a solution of paclitaxel in an organic solvent is then added and the coating composition is continuously stirred for about 6.5 hours at 60° C. or for as long as necessary for aging.

The coating composition is then sprayed onto the surface of a medical device and a heat treatment that heats the coating composition to 150° C. is applied for 16 hours or as required for densification, removal of organic residues and/or desired drug release properties.

### EXAMPLE 3

Titanium tetraisopropoxide is added drop-wise to a solution of absolute ethanol, surfactant of triblock copolymer (HO(CH₂CH₂O)₂₀(CH₂CH—(CH₃)O)₇₀(CH₂CH₂O)₂₀H) and a complexing agent acetylacetone under stirring conditions. Nitric acid was then added to the mixture. The molar ratios of the ingredients are: titanium precursor/surfactant/complexing agent/nitric acid/ethanol ¼:1:0.05:0.5:1.5:43. The final solution (pH is about 3) is stirred for 24 hours at room temperature.

The resulting coating composition is applied to the surface of a medical device and is placed an oven for solvothermal treatment at 80° C. for 18 hours and then 150° C. for 20 hours or for as long as required for densification, removal of organic residues and/or desired drug release properties.

### EXAMPLE 4

An aqueous solution containing 0.01 mol/L of titanium tetrachloride and 0.1 mol/L of hydrochloric acid is prepared. Titanium (IV) chloride is added under vigorous stirring to the aqueous solution. The aqueous solution is poured into a microwave reactor (Biotage Advancer, Biotage, Uppsala Sweden), a 0.4-MPa argon pressure is introduced into the system, and then the reactor is exposed to microwaves for 30 s at 500 Watt power level. The pressure level is maintained at a max of 1.5 bar.

An aqueous heparin solution (200 mg/10 ml water) is prepared and added under vigorous stirring to the first solution in a 1:1 ratio directly after the first solution is cooled to room temperature. Stainless steel Express Stents, Boston Scientific, were cleaned in a H₂O₂/NH₃ bath and washed in water. Stents were dip-coated 4 times in the Heparin\TiOx solution and dried in between dip-coating steps at 50° C. for 4 hours.

### EXAMPLE 5

Poly(ethylene oxide) (PEO) is dissolved in absolute ethanol by stirring and refluxing at 60° C. for 10 hours under N₂ gas flow. A mixture of Ti-isopropoxide and 2,4-pentanedione (AcAc) is dissolved in ethanol and is added into the PEO-ethanol solution followed by stirring and refluxing at 60° C. for 10 hours in N₂ atmosphere. Hydrochloric acid of 1.5 mol/L, is used as a catalyst for hydrolysis and polycondensation. The hydrochloric acid is added drop-wise into the PEO-Tiisopropoxide solution under the same atmosphere and the final solution is vigorously stirred and refluxed at 60° C. for 6 hours. The solution is aged at 60° C. for 6 to 12 hours, in N₂ atmosphere without stirring. After aging, the yellowish and transparent solution is spin coated onto a stent 10 times, and between each coating step drying is performed at 60° C. The coated stents are thermally treated at 600° C. for 1 hr., in air atmosphere.

### EXAMPLE 6

Precusors (tetraethoxysilane (TEOS), methytriethoxysilane (MTES), vinyltriethoxysilane (VTES), propyltriethoxysilane (PTES), and phenyltriethoxysilane (PhTES), ethanol, 50 mM of paclitaxel, 0.010 M HCl solution, and solid dopants are mixed and stirred to get uniform sols. The dopants used are cetyltrimethylammonium bromide (CTAB), sodium dodecyl sulfate (SDS), and hydroxypropyl cellulose (HPC). The sols containing HPC are heated to 60° C. to help dissolve the HPC. All sols are hydrolyzed in a covered beaker for one day at room temperature before 1.0 M ammonia is added to raise the pH. After gelation the gels are aged for 12 h followed by drying at room temperature for 3 days, and finally dried at 50° C. for 1 day.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

What is claimed is:

1. An implantable intravascular stent comprising:
(a) a stent sidewall structure having a surface; and
(b) a coating comprising a first metal oxide and a therapeutic agent disposed upon at least a portion of the surface, wherein the metal oxide comprises a titanium oxide or an iridium oxide.

2. The stent of claim 1, wherein the stent sidewall structure comprises a plurality of struts and a plurality of openings.

3. The stent of claim 2, wherein the coating conforms to the surface to preserve the openings of the stent sidewall structure.

4. The stent of claim 1, wherein the metal oxide is a hydrophilic titanium oxide.

5. The stent of claim 1, wherein the stent is a hydrophobic titanium oxide.

6. The stent of claim 1, wherein the first metal oxide comprises about 1 to about 80 weight percent of the coating.

7. The stent of claim 1, wherein the first metal oxide comprises about 5 to about 30 weight percent of the coating.

8. The stent of claim 1, wherein the therapeutic agent comprises an anti-thrombogenic agent, anti-angiogenesis agent, anti-proliferative agent, antibiotic agent, anti-restenosis agent, growth factor, immunosuppressant, radiochemical, or combination of thereof.

9. The stent of claim 1, wherein the therapeutic agent comprises paclitaxel, an analog thereof, a derivative thereof, or a conjugate thereof.

10. The stent of claim 1, wherein the therapeutic agent comprises sirolimus, tacrolimus, pimecrolimus, everolimus, or zotarolimus.

11. The stent of claim 1, wherein the therapeutic agent comprises about 1% to about 40% by weight of the coating.

12. The stent of claim 1, wherein the therapeutic agent comprises about or about 5% to about 30% by weight of the coating.

13. The stent of claim 1, wherein the coating further comprises a polymer.

14. The stent of claim 1, wherein the first metal oxide and the therapeutic agent are dispersed in the polymer.

15. The stent of claim 1, wherein the polymer and the therapeutic agent are dispersed in the first metal oxide.

16. The stent of claim 13, wherein the polymer comprises a block-copolymer.

17. The stent of claim 13, wherein the polymer comprises a polyether, PEBAX, a polystyrene copolymer, a polyurethane, an ethylene vinyl acetate copolymer, a polyethylene glycol, a fluoropolymer, a polyaniline, a polythiophene, a polypyrrole, a maleated block copolymer, a polymethylmethacrylate, a polyethylenetheraphtalate or a combination thereof.

18. The stent of claim 1, wherein the stent further comprises a quantity of an inorganic or ceramic oxide disposed between the surface and the coating.

19. The stent of claim 18, wherein the inorganic or ceramic oxide comprises a second metal oxide, wherein the second metal oxide comprises a titanium oxide or an iridium oxide.

20. The stent of claim 1, wherein the coating further comprises an inorganic or ceramic oxide.

21. The stent of claim 21, wherein the inorganic or ceramic oxide comprises about 1% to about 30% by weight of the coating.

22. The stent of claim 21, wherein the inorganic or ceramic oxide comprises a second metal oxide.

23. The stent of claim 22, wherein the second metal oxide comprises a titanium oxide or an iridium oxide.

24. The stent of claim 1, wherein the coating further comprises a surfactant.

25. An implantable intravascular stent comprising:
(a) a balloon-expandable stent sidewall structure having a surface and openings therein, wherein the stent sidewall structure comprises a metal; and
(b) a coating comprising a titanium oxide and an anti-restenosis agent disposed upon and adhering to at least a portion of the surface, wherein the coating conforms to the surface to preserve the opening of the stent sidewall structure.

26. The stent of claim 25, wherein the coating further comprises a polymer.

27. The stent of claim 25, wherein the stent sidewall structure comprises stainless steel.

28. An implantable intravascular stent comprising:
(a) a self-expanding stent sidewall structure having a surface and openings therein, wherein the stent sidewall structure comprises nitinol; and
(b) a coating comprising a titanium oxide and an anti-restenosis agent disposed upon and adhering to at least a portion of the surface.

29. The stent of claim 28, wherein the coating conforms to the surface to preserve the openings of the stent sidewall structure.

30. The stent of claim 28, wherein the coating comprises a polymer.
